(19) United States
(12) Patent Application Publication
(10) Pub. No.: US 2006/0010551 A1
(43) Pub. Date: Jan. 19, 2006

(54) Welding face covering

(76) Inventors: Timothy D. Bishop, Oxford, PA (US); Gary J. Thurwanger, Springfield, PA (US)

Correspondence Address: Connolly Bove Lodge & Hutz LLP, P.O. Box 2207, Wilmington, DE, 19899-2207, US

(21) Appl. No.: 11/166,811

(22) Filed: Jun. 24, 2005

Related U.S. Application Data
(60) Provisional application No. 60/588,234, filed on Jul. 14, 2004.

(51) Int. Cl. A61F 9/00

(52) U.S. Cl. 2/15

(57) ABSTRACT

A welding face covering such as a digital welding helmet includes a face protector which would be located in front of the welder's eyes. The inner surface of the face protector has a digital viewing screen. A digital lens is mounted externally of the face protector for viewing the welding site. The digital lens transmits an image of the welding site to the viewing screen. As a result, the welder can view the welding site by looking at the screen rather than looking through a viewing window of the face covering or helmet.

### CROSS-REFERENCE TO RELATED APPLICATION

This application is based upon provisional patent application Ser. No. 60/588,234, filed Jul. 14, 2004.

### BACKGROUND OF THE INVENTION

The present invention is related to welding face coverings such as welding helmets which would be used under conditions where it is sometimes difficult for the welder to clearly view the welding site. It is known to provide various techniques to assist a welding operator being able to view the welding site. Traditionally fixed shade filter lenses have been used. More recently, LCD filters have been used which electronically darken in the presence of a welding arc, known as an ADF (automatic darkening filters) to assist the welder. Generally, such ADF welding helmets include a fixed shad IR and UV viewing window with a variable shade luminous lens, through which the welder would look while performing the welding process. It is also known to provide various types of controls to optimize the shade and switching adjustments of the ADF.

### SUMMARY OF THE INVENTION

An object of this invention is to provide a welding face covering which utilizes techniques for assuring proper viewing of the welding site by the welder and maximizes protection to the welder.

A further object of this invention is to provide such a welding face covering that can be conveniently operated so as to maximize the ability of the welder to view the welding site during the welding process.

In accordance with this invention, the welding face covering includes a face protector that would be disposed in front of the users eyes. The protector would preferably be mounted to the head of the user, such as being part of a welding helmet or goggles or face shield. The face protector itself in the area of the welders eyes, could be opaque. The wearer however would be able to see the welding site through the use of a remote digital lens which would be directed toward and view the welding site and then would send a digital image of the welding site to a viewing screen on the inside of the face protector in the general vicinity of the user's eyes.

In one practice of the invention, the remote digital lens could be mounted on the external surface of the welding helmet or shell. An alternative location would be to mount the lens on the welding rod holder. The invention may be practiced with the use of a control unit such as a programmer to control various parameters of the viewing lens such as the shading, focusing, magnification, zooming, contrast, brightness, etc. The control unit could be mounted to the welding helmet or be clipped to the welder's pants or belt or any other clothing part or could be in any other desired location including being mounted on the welding rod holder as part of the same structure for the digital lens. The invention could also allow welding in dark or poorly lighted areas. The digital viewing area could even be programmed to color-code the weld material based on temperature, using IR sensors.

### The Drawings

FIG. 1 is a front elevational view of a welding helmet in accordance with this invention;

FIG. 2 is a side elevational view of the welding helmet shown in FIG. 1;

FIG. 3 is a side elevational view of a modified form of welding helmet in accordance with this invention;

FIG. 4 is a front elevational view showing an alternative practice of this invention in the form of welding goggles;

FIG. 5 is a side elevational view showing a control unit which may be used in accordance with this invention; and

FIG. 6 is a side elevational view showing a welding rod holder in accordance with this invention.

### DETAILED DESCRIPTION

The present invention is generally directed to providing a welding face covering which includes a face protector that would be mounted in front of the user's eyes. Such face covering could be of any desirable form such as a welding helmet, welding goggles, including goggles incorporated in a welding hood, or a face shield. The invention is characterized by permitting the welder to view the welding site through the use of an externally mounted digital lens which would be directed toward and would view the welding site. The digital lens sends a digital image of the welding site to a viewing screen on the inner surface of the face protector. Thus the welder's eyes are completely shielded and protected and yet the welder is readily able to see the welding site and perform the welding operation.

A preferred practice of the invention generally involves a welding helmet designed with a digital viewing screen inside of the helmet. The entire helmet could be opaque to at least an ANSI shade #14. This electronic viewing screen would be fed a digital image of the welding from a remote electronic lens source positioned either on the helmet's outside shell or on the welding rod holder. No dangerous direct optical radiation would pass through the helmet. A control module would allow the welder to select or adjust any desired parameters of the viewing screen. The electronics of this assembly could either be battery or solar powered.

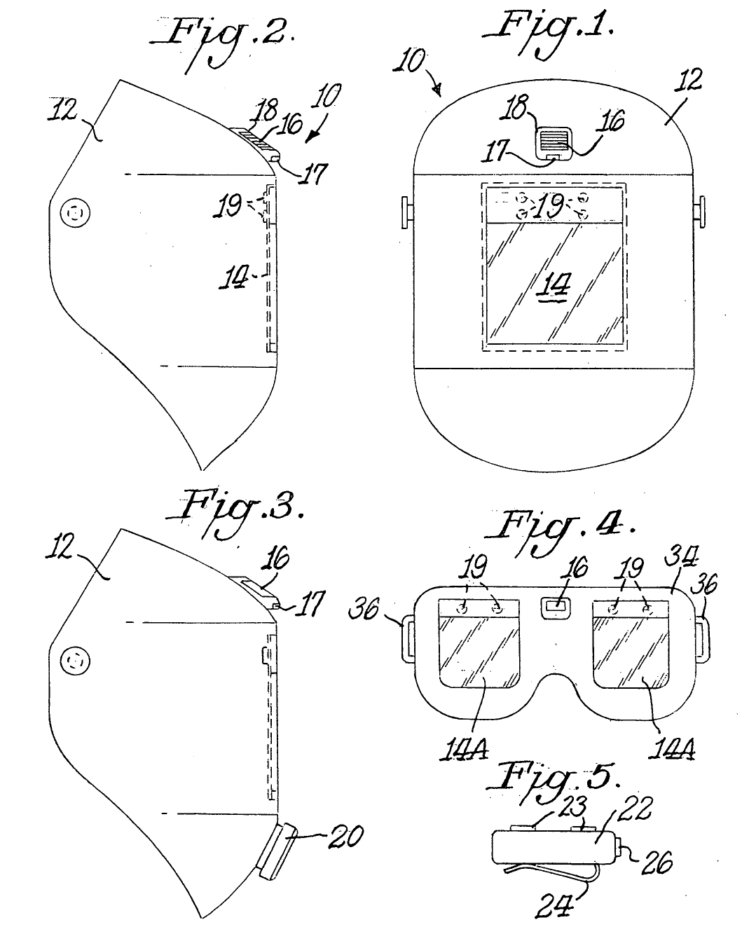

FIGS. 1-2 illustrate a face protector 10 in accordance with one practice of this invention. As shown therein, the face protector 10 is in the form of a standard type welding helmet 12 which could be of generally known construction in the sense of the materials used for making the helmet and in the sense of the structure used for mounting the helmet on the head of the welder. Thus the helmet has a front section and side sections. The head mounting structure would be located at the side sections and could be of known construction. One of the features of welding helmet 12 is that the section of the shell located in the front of the helmet in the vicinity of the user's eyes, would have a viewing screen 14 mounted on its inner surface. This portion of the helmet may be considered as a face protector which would shield the welder's eyes. If desired, the outer surface of the shell at the location of the face protector could be opaque because the welder would be able to view the welding site by looking at the viewing screen 14.

In accordance with this invention, a digital lens such as lens 16 is mounted externally on the face protector at some remote location. In the illustrated embodiment of FIGS. 1-2, the lens 16 is mounted directly to the outer surface of the shell forming the welding helmet 12. The digital lens 16 would be located so as to be directed toward and thereby view the welding site. The digital lens 16 would send a digital image of the welding site to the viewing screen 14. Lens 16, for example, could include a transmitter 17, or utilize an electrical wire to send the image to a receiver on screen 14. As a result, the wearer of the helmet 12 can readily see the welding site by looking at the viewing screen 14 and it is not necessary to form any viewing window in the front surface, i.e. the face protector portion, of the helmet 12. Although the invention could be broadly practiced with only a single digital lens, the invention could also be practiced with a plurality of digital lenses located in a cluster adjacent to each other, or in sets of digital lenses mounted at any suitable locations. As illustrated, the digital lens 16 preferably includes a protective cover 18.

While FIGS. 1-2 illustrate the basic structure of a welding helmet 10 in accordance with the practice of this invention, other forms of welding helmets could also be used. FIG. 3 for example, shows a known type of respirator 20 to be incorporated in welding helmet 12.

The invention is preferably practiced with the use of a control unit which would optimize the viewing parameters on the viewing screen 14. FIG. 5 for example, illustrates a control unit in the form of a program control module 22 which could control the electronic adjustment of such parameters as shade number, focusing, magnification, zooming, contrast, brightness, etc. The control module 22 may be of any known construction that could control the function of the viewing screen or lens.

The control module 22 could include a key pad 23 having buttons which can be manipulated to select the different parameters which would be controlled. The control module 22 includes a transmitter 26 or electrical wire to send its signal to a receiver on screen 14. Through use of the clip 24 or through the use of any other type of fastener such as a strap the electronic control module 22 could be mounted to any suitable part of the welder's clothing. Alternatively, the control module could be at any other suitable location such as on the welding helmet or electrode holder.

The invention provides a number of distinct benefits. For example, there would be 100% protection of the eyes at all times. No switch-over time delay would be necessary. As noted, the parameters of the viewing could be controlled through the use of a program. The desired electronic adjustment could be made in advance in accordance with the type of welding operation being performed. The control module could be of any suitable form and could be located in any suitable place. FIG. 5 for example, illustrates the control module 22 to include a spring clip 24 so that the control module 22 could be worn on the hip of the wearer such as being clipped to the user's pants or belt, or being mounted to the outside of the welding helmet, or to the rod holder. Operator feedback could be displayed on the screen.

The invention provides a viewing area isolated from the welding operation. This could allow welding in areas or locations that are not normally viewable. Thus as shown in FIG. 6, the lens 16 could be incorporated on a welding rod holder 28 which could otherwise be of conventional construction. If desired, the control module could also be incorporated on the welding rod holder 28 and could include a program selector such as joystick 32 mounted to holder 28. The invention could be practiced in any welding procedure and thus the invention could be practiced where the control module is mounted at any suitable location such as being mounted on a MIG or TIG wire welder.

The invention can be practiced where, for example, one set of digital lenses is mounted to the helmet 12 and another set is mounted to the holder 28. Similarly, sets of lenses 16 could be located at different portions of the helmet 12. The location would be such so that when the user is facing the welding site the lenses 16 would be disposed to be in the line of view of the welding site.

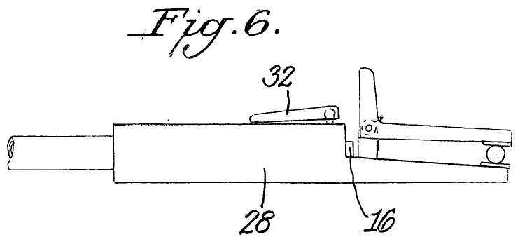

Although FIGS. 1-3 show the welding face covering to be in the form of a welding helmet, the invention could be practiced with other types of welding face coverings. FIG. 4, for example, illustrates goggles 34 which would include a pair of screens 14A, 14A. The goggles could include attachment loops 36 for receiving a strap to mount the goggles around the users head or the goggles could be incorporated as part of a welding hood. Instead of goggles, the invention could also be practiced with known types of face shields. What is important is that a face protector would be provided which would be located in front of and protect the welder's eyes. The face protector however, would include a viewing screen on its inner surface so that the welder could readily and accurately view the welding site or image while the eyes are being protected or shielded.

If desired, various types of controls could be mounted to the welding face covering at the viewing screen 14. Thus, FIGS. 1-2 show manual controls or knobs 19 inside the helmet 12 for controlling various parameters such as increasing the intensity for different types of welding programs and/or for magnifying the image. These controls could be of the type controlled by the program of module 22. In that sense, the manual controls are part of a control unit. Alternatively, the manual controls could be in addition to and separate from the electronic controls performed by the program of module 22.

Where the invention is practiced through the use of goggles 34 a screen 14A is provided for each of the welder's eyes. Each screen 14A may include its own manual controls 19, in addition to operating in conjunction with an electronic control module 22.

The invention provides the ability to record the image and thereby document weld quality. For example, in certain critical areas such as in the aerospace industry, it is essential to keep precise records of all aspects of the operation so as to provide the ability to detect how later occurring problems might have arisen. By having the ability to accurately view the welding site through use of the invention, precise documentation could be made as the welding operation proceeds.

The invention also lends itself as a training tool since multiple users could view the same image. The invention could also be used as a production-monitoring tool.

Because the image being viewed on the screen 14 is on the inside of the welding face covering or helmet 12, it is possible to use a lighter weight helmet by eliminating cover plates and large area filters which are used in prior known ADF welding helmets.

The invention also has the advantage of eliminating the need for infra-red or ultraviolet structure used in the prior art for filtering. The invention could use a very small replaceable cover lens to protect the digital lens.

The use of the digital lenses and the digital viewing screen lends itself to improved color recognition of image at the welding site. The electronics from the control module 22 could be programmed to reduce the brightness of the welding arc and thereby be able to better view the surrounding areas of the weld. The electronics could be programmed to be zoomed in to start the weld and then to pan back automatically as the weld is started. The electronics could be used to be programmed to follow the weld arc as it is moved.

Depending on the degree of quality and sophistication desired, the image of the weld site on the screen 14 could be a black and white image for lower cost or could be a color image for a truer viewing of the actual welding site. The image could also be programmed to be color-coded to show the temperatures of the metals through the use of an infrared temperature sensor.

The invention thus provides a marked improvement over prior techniques for viewing the welding site during a welding procedure.

What is claimed is:

1. A welding face covering comprising a face protector for being disposed in front of the wearer's eyes, said face protector having an inner surface and an outer surface, mounting structure for mounting said face protector to the head of the wearer to dispose said inner surface of said face protector directly in front of the wearer's eyes, a digital viewing screen on said inner surface, a digital lens external of said face protector for being directed toward and viewing the welding site, and said digital lens sending a digital image of the welding site to said viewing screen whereby a wearer may view the welding site by looking at said viewing screen.

2. The covering of claim 1, wherein said face protector is sufficiently opaque to prevent the wearer from viewing the welding site other than by viewing said screen.

3. The covering of claim 1, wherein said face protector is part of a welding helmet.

4. The covering of claim 3, wherein said welding helmet comprises a shell having a front section and side sections, said face protector being at said front section, and said lens being mounted to and externally of said shell.

5. The covering of claim 4, including a cover mounted at said lens.

6. The covering of claim 3, wherein said helmet includes a respirator.

7. The covering of claim 3, including a control unit for controlling the viewing parameters of the welding site.

8. The covering of claim 7, wherein said viewing parameters are selected from the group consisting of shade number, focusing, magnification, zooming, contrast, brightness and combinations thereof.

9. The covering of claim 7, wherein said control unit comprises an electronic program module.

10. The covering of claim 9, wherein said electronic control program module includes a keypad for selecting the control parameters.

11. The covering of claim 9, wherein said electronic control program module is built into said viewing screen.

12. The covering of claim 9, wherein said control unit further includes a manually operable control unit.

13. The covering of claim 12, wherein said manually operable control unit is mounted at a location consisting of said viewing screen, said helmet, a wire welder, and a rod holder.

14. The covering of claim 9, wherein said program module includes a mounting member to mount said program module to clothing of the user or to said helmet.

15. The covering of claim 3, wherein said face protector is sufficiently opaque to prevent the wearer from viewing the welding site other than by viewing said screen.

16. The covering of claim 2, in combination with a welding rod holder, and said lens being mounted to said welding rod holder.

17. The covering of claim 1, wherein said face protector is part of welding goggles.

18. The covering of claim 17, wherein said welding goggles includes one of said viewing screens for each of the wearer's eyes.

19. The covering of claim 17, wherein said goggles is part of a hood.

20. The covering of claim 1, wherein said face protector is part of a face shield.

21. A method of viewing a welding site comprising the steps of disposing a face protector in front of a welder's eyes, locating a digital viewing screen on the inner surface of the face protector directly in front of the welder's eyes, and the welder viewing the welding site by use of a digital lens mounted externally of the face protector which sends a digital image of the welding site from the digital lens to the viewing screen.

22. The method of claim 21 wherein the face protector is part of a welding helmet which has an opaque front portion, and the welder viewing the welding site solely by looking at the viewing screen.

23. The method of claim 21 including mounting the digital lens on a welding rod holder.
